(19) United States
(12) Patent Application Publication
(10) Pub. No.: US 2025/0049615 A1
(43) Pub. Date: Feb. 13, 2025

(54) QUICK APPLICATION OF VENTED TOTAL CONTACT CAST

(71) Applicant: Alessandro BARBERIO, Aurora (CA)

(72) Inventor: Alessandro BARBERIO, Aurora (CA)

(21) Appl. No.: 18/721,727

(22) PCT Filed: Oct. 7, 2022
(86) PCT No.: PCT/CA2022/051487

§ 371 Date: Jun. 19, 2024

Related U.S. Application Data
(60) Provisional application No. 63/361,348, filed on Dec. 20, 2021.
(60) Provisional application No. 63/372,332, filed on Mar. 4, 2022.
(60) Provisional application No. 63/408,304, filed on Sep. 20, 2022.

(51) Int. Cl. A61F 13/04; A61F 5/058

(52) U.S. Cl. CPC A61F 13/046; A61F 5/0585

(57) ABSTRACT

A vented total contact cast for a wound on a patient's leg features ventilation members in the form of adhering, compressible flexible and ventilating 3D fabric spacers with release liners which cover an adhesive configured for attaching to a stockinette to be received over the patient's leg. The ventilation members may be provided in sets of different shapes and different dimensions, for selective mounting to the stockinette based on the area of the leg to be aerated and the size of the leg.

### FIELD OF THE INVENTION

The present invention relates generally to an orthopedic vented total cast, and more particularly, in some arrangements, to such a cast with plural components, for example four, arranged to (i) support a patient's foot and leg while offloading the weight from a wound on the leg; (ii) reduce internal edema pressure; and (iii) provide atmospheric aeration.

### BACKGROUND

Diabetic foot ulcers are a global problem, for instance for people with diabetes who suffer from poor blood flow and as such develop sores and open wounds on their feet. Such sores or ulcers negatively affect the individual's lifestyle including limiting mobility functions and costing expensive recoveries. In some cases, diabetic foot ulcers can lead to amputation that can result in heavy financial burden on the healthcare system.

Off-loading the patient's weight from the wound is today an attractive option, which can be achieved by applying a total contact cast to the patient's leg.

Available application methods are not completely effective because their use is time consuming and costly to the patient and to the health system.

Some of their common shortcomings include (i) being relatively hard to put on and to take off, uncomfortable, non-breathable; (ii) ineffective at offloading or adapting weight from open wounds; and (iii) suffocating pressure by edema pressure, could cause wounds with burns during removal of casts.

Challenges still exist. Relatively, it would be an improvement in the art to replace current techniques with other technologies.

### SUMMARY OF THE INVENTION

The present invention relates to an orthopedic total contact cast system that can be used for treatment of diabetic foot ulcers on legs and on feet. Instead, the described total contact cast can be used to support a patient's foot leg, while offloading weight and distribute such weight to other parts of the foot or and leg.

The described total contact cast includes two or more fiber glass cast rolls, underlayments as sterilized cotton, a tubular stockinette, optionally a ventilated plantar foot. A series of compressible 3D fabric spacers to absorb the edema pressure, and to ventilate the wounded leg.

According to an aspect of the present invention there is provided a system for a total contact cast to treat a wound on a patient's leg comprising:
a wound dressing configured for application to the wound;
a fluidically permeable stockinette configured to receive the patient's leg with the wound dressing on the wound;
at least one ventilation member arranged for mounting to the stockinette, wherein said at least one ventilation member respectively comprises a body of three dimensional (3D) fabric spacer material forming therein a pathway for air flow from an ambient environment to the patient's leg; and
cast material for forming an outermost encapsulating layer of the total contact cast;
wherein the at least one ventilation member respectively comprises one or more adhesive areas on a common side of the ventilation member and removably covered for adhesively mounting to the stockinette.

This provides an arrangement of total contact cast which can be more quickly and simply formed or assembled.

In one arrangement, the at least one ventilation member comprises a selected one of a plurality of prefabricated ventilation members having a common shape and different dimensions.

According to another aspect of the invention there is provided a kit for forming a total contact cast to treat a wound on a patient's comprising:
a wound dressing configured for application to the wound;
a fluidically permeable stockinette configured to receive the patient's leg with the wound dressing on the wound;
at least one aeration arranged for mounting to the stockinette, wherein said at least one ventilation member respectively comprises a body of three dimensional (3D) fabric spacer material forming therein a pathway for air flow from an ambient environment to the patient's leg; and
cast material for forming an outermost encapsulating layer of the total contact cast;
wherein the at least one ventilation member respectively comprises an attachment device supported on one side of the ventilation member and configured to mount to the stockinette;
wherein the at least one ventilation member comprises at least one set of prefabricated ventilation members having a common shape and different dimensions for selective application to the patient's leg.

This provides an arrangement of total contact cast which can be more quickly and simply formed or assembled.

In one arrangement, when the at least one ventilation member respectively comprises a lower leg ventilation member arranged to cover a lower leg portion of the patient's leg excluding a foot thereof, and when the lower leg ventilation member is longitudinally elongated to have longitudinally opposite ends and transversely opposite sides which are longer in length than the ends, so as to be arranged to be oriented in a lengthwise direction of the patient's leg between the foot and a hip, the lower leg ventilation member further includes a preliminary mounting member located at one of the ends and substantially transversely centrally thereof, wherein the preliminary mounting member is configured to attach to the stockinette for positioning the lower leg ventilation member in relation to the lower leg portion of the patient's leg.

Preferably, the preliminary mounting member comprises removably covered adhesive so as to be configured to attach to the stockinette by bonding.

In one arrangement, when the at least one ventilation member respectively comprises a lower leg dorsal ventilation member arranged to cover a dorsal lower leg portion of the patient's leg between the knee and the foot of the patient's leg, and when the lower leg dorsal ventilation member is longitudinally elongated to have longitudinally opposite ends and transversely opposite sides which are longer in length than the ends, the lower leg dorsal ventilation member further includes an integral tailpiece at a bottom one of the ends arranged to be in proximal relation to the foot, the tailpiece is elongated relative to a longitudinal direction of the lower leg dorsal ventilation member from the bottom end to a distal end of the tailpiece, and the tailpiece is narrower in width relative to a transverse direction than the body of 3D fabric spacer material forming the lower leg dorsal ventilation member.

In one arrangement, the at least one set of prefabricated ventilation members comprises a plurality of the sets having different shapes for mounting to different areas of the patient's leg.

In one arrangement, the at least one ventilation member respectively includes one or more adhesive areas on a common side of the ventilation member and removably covered for adhesively mounting to the stockinette.

According to yet another aspect of the invention there is provided a method for forming a total contact cast to treat a wound on a patient's leg comprising:
applying a wound dressing to the wound;
after applying the wound dressing, covering the patient's leg with a stockinette;
after covering the patient's leg with a stockinette, mounting one or more ventilation members respectively comprising three dimensional fabric spacer material to the stockinette; and
after mounting one or more ventilation members, encapsulating in cast material;
wherein mounting one or more ventilation members to the stockinette comprises bonding each of the ventilation members to the stockinette.

This arrangement provides a quicker and simpler manner of forming a total contact cast by bonding using existing adhesive applied to the ventilation member, that is a premanufactured component, instead of using fastening mechanisms or devices distinct from the ventilation member to secure the same to the patient's leg.

In one arrangement, mounting one or more ventilation members to the stockinette comprises selecting each of the ventilation members from a prefabricated set having a common shape arranged to conform to a prescribed portion of the patient's leg and different dimensions.

In one arrangement, mounting one or more ventilation members to the stockinette comprises respectively aligning said one or more ventilation members relative to the patient's leg using a first adhesive portion at one end of a respective one of the ventilation members and, after aligning, respectively securing said one or more ventilation members using a second adhesive portion covering a larger surface area of the respective ventilation member than the first adhesive portion.

According to yet another aspect of the invention there is provided a method for forming a total contact cast to treat a wound on a patient's leg comprising:
applying a wound dressing to the wound;
after applying the wound dressing, covering the patient's leg with a stockinette;
selecting, for each area of the patient's leg to be aerated, one of a plurality of ventilation members of a prefabricated set with a common shape arranged to conform to a prescribed portion of the patient's leg and different dimensions;
after covering the patient's leg with a stockinette, mounting the selected ventilation member to the stockinette; and
after mounting one or more ventilation members, encapsulating in cast material.

This arrangement provides a quicker and simpler manner of forming a total contact cast by provision of prefabricated components from which suitable components may be selected.

In one arrangement, mounting one or more ventilation members to the stockinette comprises mounting at least one of said one or more ventilation members in a stretched condition over protruding portions of the patient's leg.

In one arrangement, mounting one or more ventilation members to the stockinette comprises bonding each of the ventilation members to the stockinette.

It will be appreciated that each of the foregoing features are independent of one another and can be combined.

### BRIEF DESCRIPTION OF THE DRAWINGS

The invention will now be described in conjunction with the accompanying drawings in which:

FIG. 1 represents a leg to receive a total contact cast;

FIG. 2 represents a diabetic foot ulcer;

FIG. 3 represents a wounded care dressing ready to be applied on top of the foot ulcer;

FIG. 4 depicts the wound care dressing on the diabetic foot ulcer of FIG. 1;

FIG. 5 depicts the leg being covered by a tubular stockinette and folded upwards towards the phalanges;

FIG. 6 depicts the leg with 3D fabric spacers, release liners, adhesive strip under the liners for adherence to the stockinette, and a narrow 3D fabric spacer strip at the bottom of one of the 3D fabric spacer;

FIG. 7 depicts leg with one of the 3D fabric spacers of FIG. 6 ready to be adhered on the stockinette;

FIG. 8 depicts a continuation of FIG. 7 where it shows the leg being covered frontally by one of the 3D fabric spacers;

FIG. 9 shows the leg with dorsal 3D fabric spacer ready to be adhered on the calf and an optional stripe to vent the plantar base of the foot;

FIG. 10 represents the 3D fabric spacers ready to be adhered on the malleoli of leg;

FIG. 11 shows 3D fabric spacers receiving atmospheric air;

FIG. 12 shows a venting sole;

FIG. 13 shows a protruded base with protrusions with longitudinal channels;

FIG. 14 shows vented leg covered by a layer of cotton;

FIG. 15 shows leg immobilized by a total contact cast via three layers of fiber glass cast;

FIG. 16 shows a vented total contact cast by air;

FIG. 17 shows leg inside a total contact cast ready to be installed inside a walking shoe;

FIGS. 18 to 21 show another arrangement of total contact cast for application to a patient's foot/leg; and

FIGS. 22 to 24 show an ventilation member of an arrangement of total contact cast in end, plan and cross-sectional views; and

FIG. 25 shows sets of ventilation members forming part of a kit for forming a total contact cast, according to an aspect of the present invention.

In the drawings like characters of reference indicate corresponding parts in the different figures.

### DETAILED DESCRIPTION

The accompanying figures show a total contact cast for diabetic foot ulcers. The total contact cast generally comprises a wound care dressing, a tubular stockinette, compressible flexible and ventilating 3D fabric spacers, a stepping base with venting protrusions under a perforated sole, cast rolls of fiberglass or plaster of Paris, medical cotton rolls, and a 3D fabric spacer venting stripe anchored to the posterior side of the leg.

A diabetic foot ulcer has to be taken care off via debridement, medicating, offloading, atmospheric ventilating, and decreasing internal suffocating edema pressure.

In one arrangement of total contact cast, the precut series of fabric spacers are prepacked or precontained.

In one arrangement of total contact cast, the 3D fabric spacer comprises a compressible, venting 3D fabric spacers arranged to aerate the wounded leg.

Elastic 3D fabric spacers adapt to limb's curvatures.

For example, 30 fabric spacers are pre boxed in all dimensions.

3D fabric spacers act to absorb the edema's compressive suffocating pressure.

A stepping walking base with venting protrusions is coupled to a perforated sole.

Rolls of immobilizing casts act to off load the patient's weight on the open wound.

Sterilized cotton act to sanitize internally the fiberglass cast.

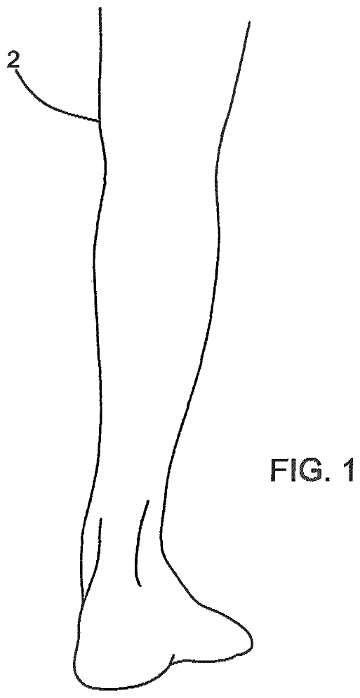

FIG. 1 represents a wounded leg as in 2 at its plantar surface not visible to receive medical treatment and a total contact cast.

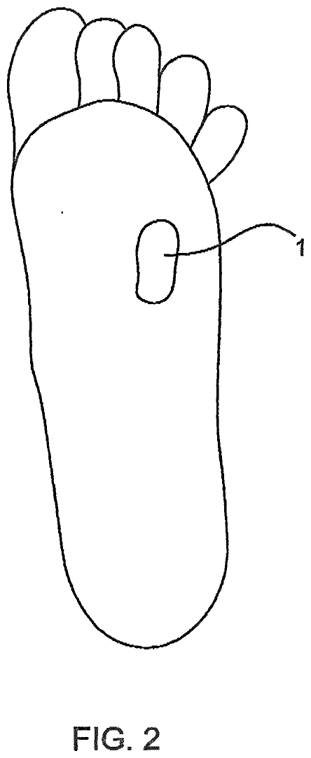

FIG. 2 represents a diabetic foot ulcer as in 1, which is ready for debridement.

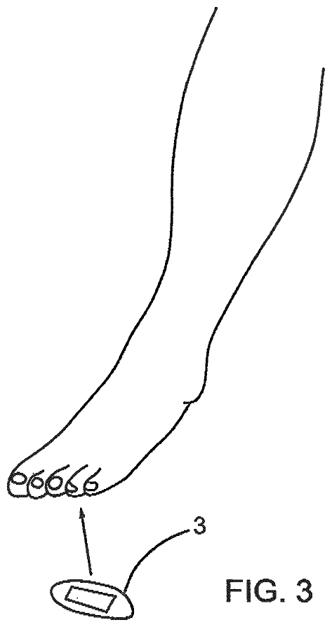

FIG. 3 represents a wound care dressing as in 3 ready to be applied on the foot ulcer 1. The dressing 3 can be optionally medicated, offering various chemical and physical properties.

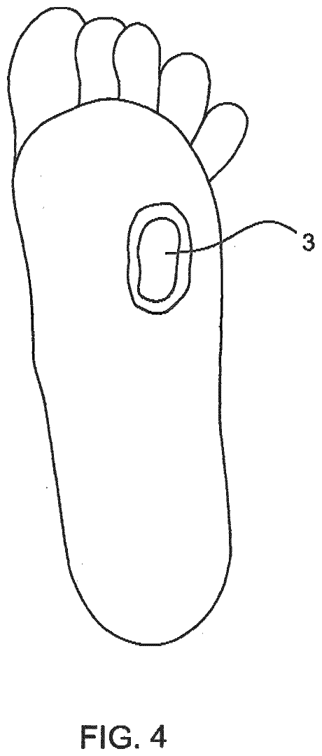

FIG. 4 depicts the wound care dressing as in 3 on top of the diabetic foot ulcer 1 of FIG. 2.

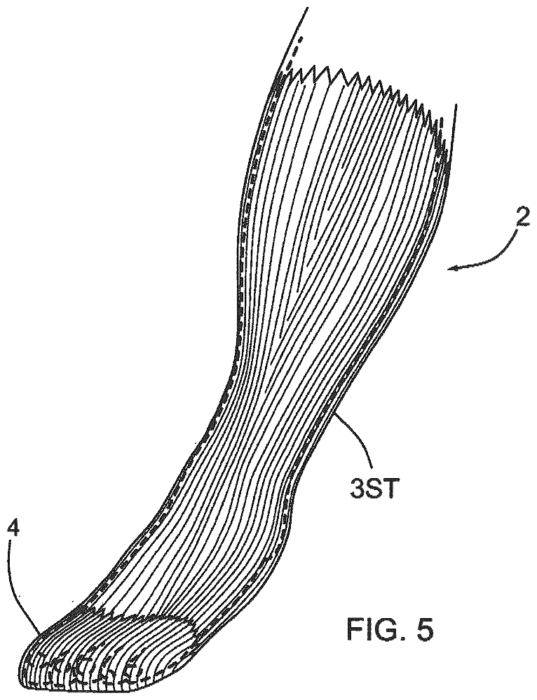

FIG. 5 depicts leg 2 covered by tubular stockinette as in 3ST and folded upwards as in 4 towards the phalanges.

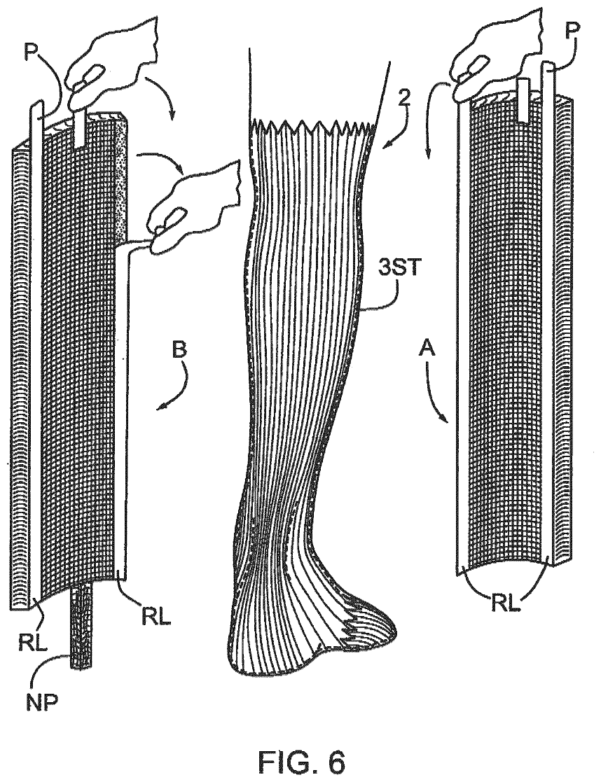

FIG. 6 depicts the leg as in 2 with 3D fabric spacers as in A and in B with release liners as in RL. By pulling each RL via longitudinal portions as in P a stripe of adhesive nature will adhere A and B to stockinette 3ST.

At the bottom of 3D fabric spacer B extends a narrow portion of same fabric spacer B as NP. The NP portion is extended optionally further down than the foot's heel in order to transmit laterally atmospheric air to the foot's base.

NP is used optionally if a plantar venting base is required.

A and B are prepacked in groups of different sizes, thicknesses, elongation properties, compressive forces, weights, flexibility, degree of sterility level and colours.

After applying covers A and B it remains a small gap between them in order to have the freedom to install narrower ones and avoid juxtapositions.

Edema's compressibility allows the 3D fabric spacer to absorb the suffocating pressure exercised by the edema from the wounded limb.

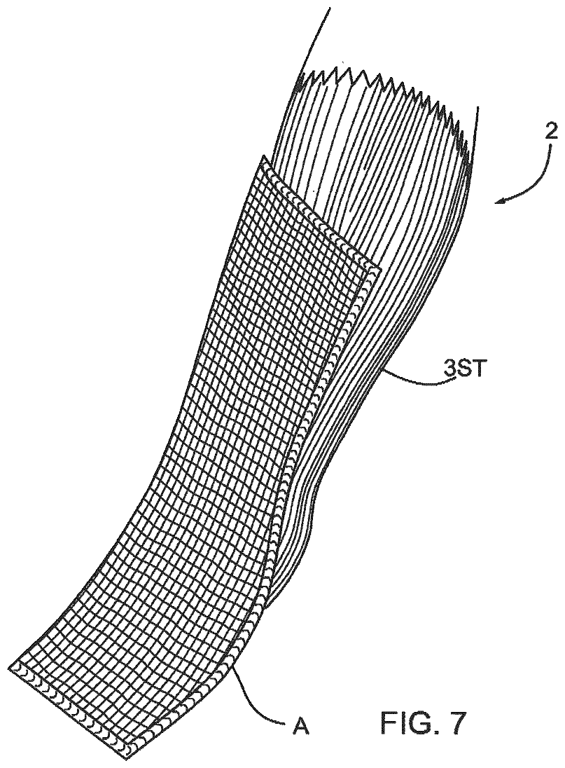

FIG. 7 shows leg 2 with frontal 3D fabric spacer as in A ready to be adhered on the stockinette as in 3ST.

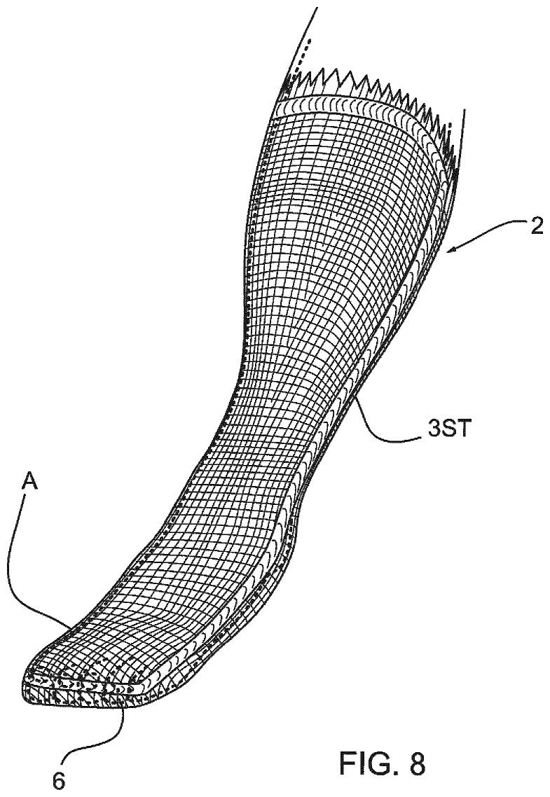

FIG. 8 shows a final continuation of FIG. 7. It depicts leg 2 being covered frontally by a 3D fabric spacer as in A.

Cover A is adhered laterally and slowly to stockinette 3ST after removal of liners RL as in FIG. 6.

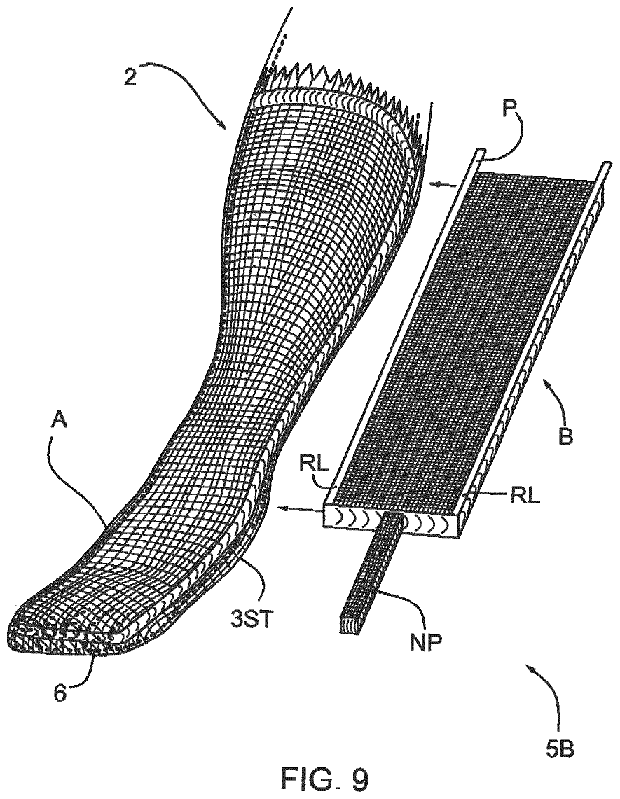

FIG. 9 shows leg 2 with dorsal 3D fabric spacer as in B ready to be adhered on the stockinette 3ST by pulling out portion P of release liners as in RL.

To be used only if the venting base will be installed exist.

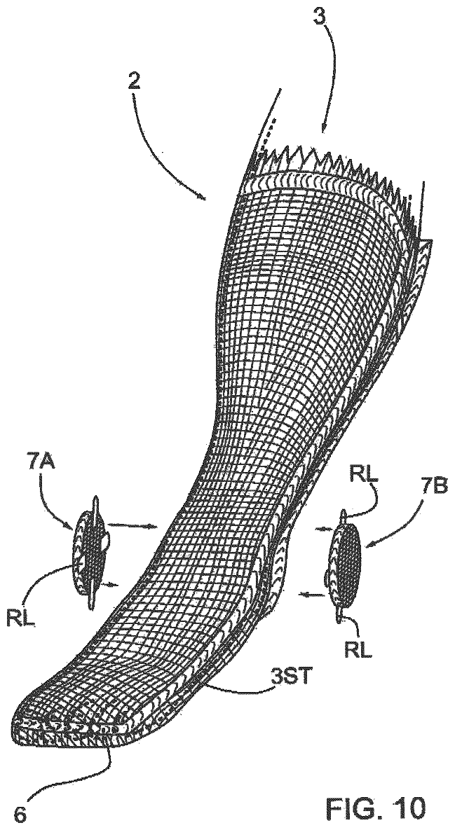

FIG. 10 represents the circular or oval 3D fabric spacers as in 7A and as in 7B to be adhered on the malleoli of leg 2 by removing the four release liners as in RL.

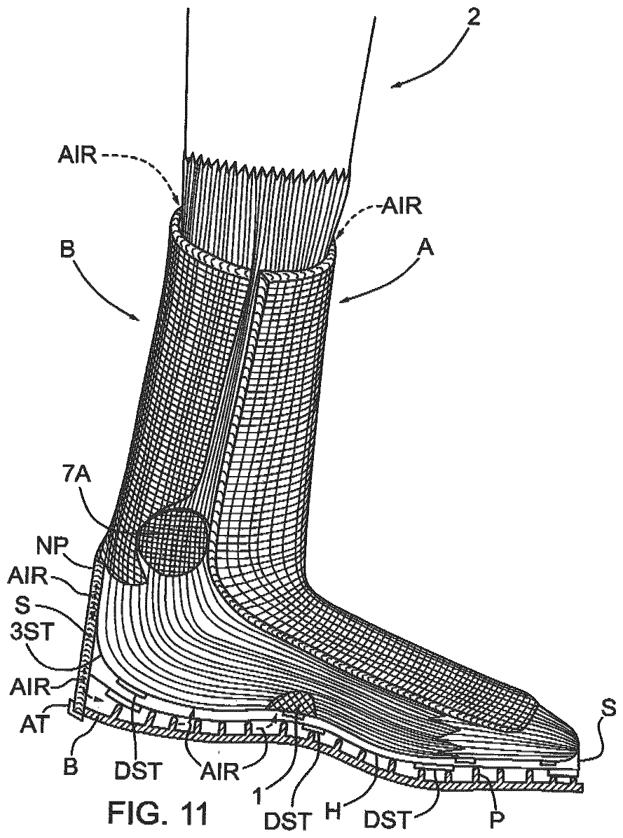

FIG. 11 allows the surgeon to apply the total contact cast in a quicker and in a simplified fashion by using prepacked 3D fabric spacer units. Covers A, B, 7A and 7B be applied only in five minutes by a well-trained care giver.

The application of 3D fabric spacers represents a total atmospheric ventilation towards the injured leg as in 2 in order to dissipate excessive heat, bacteria, sweat, edema and edema pressure. Thus improving the quality of rehabilitation for the diabetic population.

The application of 3D fabric spacers reduces the number of applications of additional total contact casts.

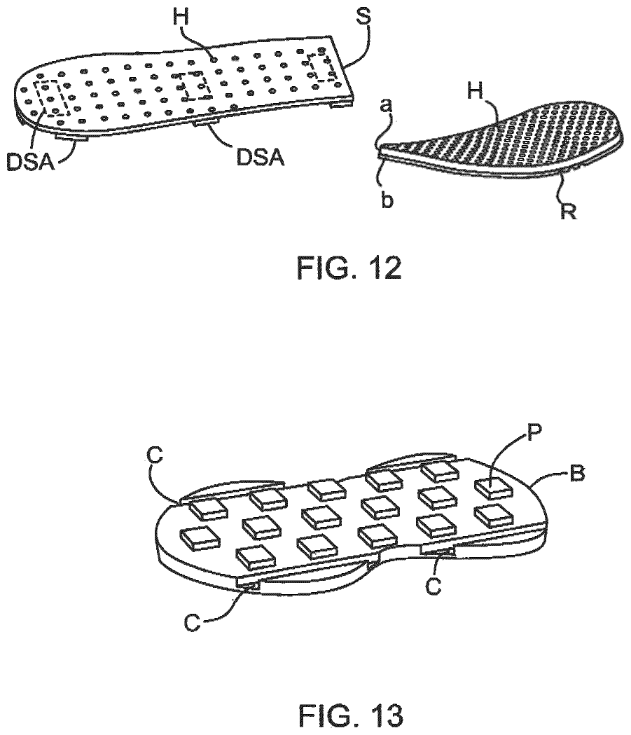

Sole S is multiperforated as shown in FIG. 12 by holes as in H to allow penetration of air as in AIR.

Sole S and base B are very flexible and adapt to the curves of the plantar base smoothly as soon as the immobilizing cast is applied.

Sole S and base B are prepacked as a single unit.

Sole S and base B are attached to each other by double sided tapes as in DST. They are offered for left and right sided. They could be custom made.

Sole S is attached to the plantar portion of the foot on the stockinette as in 3ST via double sided tapes as in DST.

The venting base as in B with its perforated sole as in S is an optional application.

Base B has protrusions as in P. They could be square, circular, rectangular able to support the patient's weight without collapsing.

Air as in AIR travels between protrusions P and reaches the plantar base of the foot via the holes H of sole S to avoid maceration and excessive accumulation of serous fluids.

Atmospheric air as in AIR penetrates the total contact cast from its upper circular edges and travels downwards towards Achille's tendon until it penetrates the longitudinal portion as in NP. Then AIR coming from NP will enter between protrusions P of base B until it refreshes the plantar portion of leg 2.

Narrow portion NP is attached externally to base B by adhesive as in AT.

FIG. 12 depicts the sole S of FIG. 11 in a diversified fashion as it follows: It depicts sole S with a series of venting holes as in H and equipped with ridges as in R.

It could be made with a dual hardness to offer more strength and comfort.

Layer a is soft enough to accommodate comfortably the wounded ulcer.

Layer b is hard enough to support the patient's weight.

Sole S is attached to base B via double sided adhesives as in DSA.

FIG. 13 has channels in many directions as in C to allow more flexibility towards the foot curves.

Protrusions P are larger than holes H sole S to offer more safety to the wounded area.

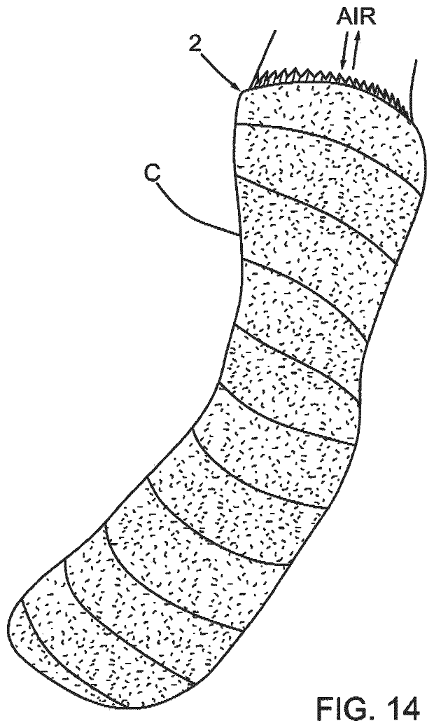

FIG. 14 describes leg 2 covered by a layer of cotton as in C to absorb bad smells, gases and provide a comfortable cushioning for the swollen skin by the edema pressure.

Cotton layer C shields the skin from the cutting saw during decasting of fiberglass cast.

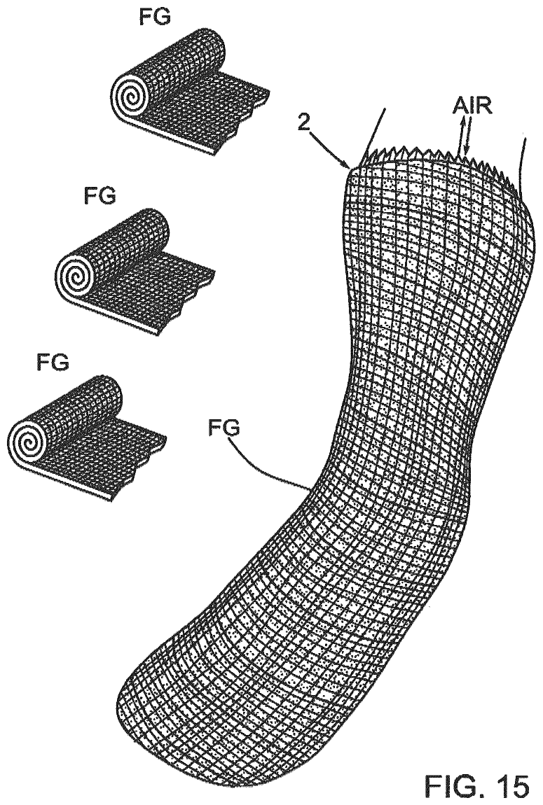

FIG. 15 depicts three rolls of fiber glass cast as in FG.

FIG. 15 shows leg 2 inside a total contact cast immobilized by FG fiberglass cast, with atmospheric air as in AIR penetrating the cast from its upper circular side.

Air as in AIR ventilates the wounded skin reducing itchiness and maceration.

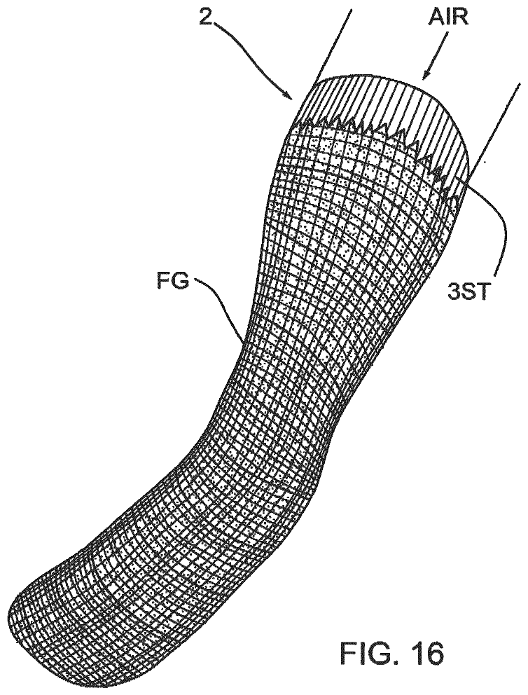

FIG. 16 shows a total contact cast fully assembled on leg 2 by fiber glass cast as in FG with tubular stockinette as 3ST overlapping the FG at its top circumference.

The overlapping portion of 3ST protects the skin from being injured by the FG cast when the patient is walking.

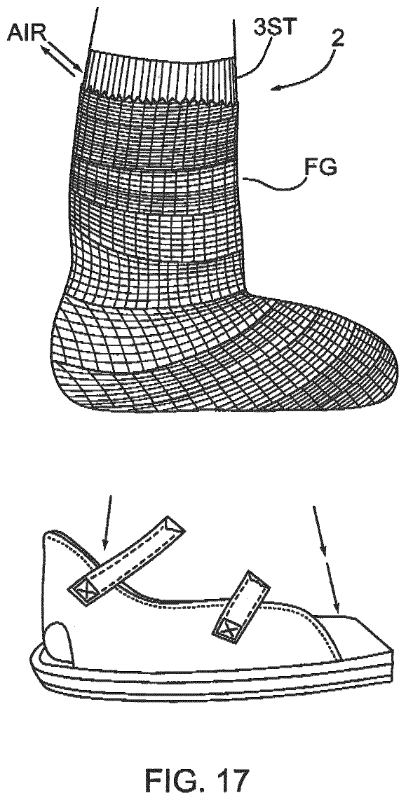

FIG. 17 shows the immobilized leg as in 2 inside a total contact cast ready to be installed inside an orthopedic walking shoe.

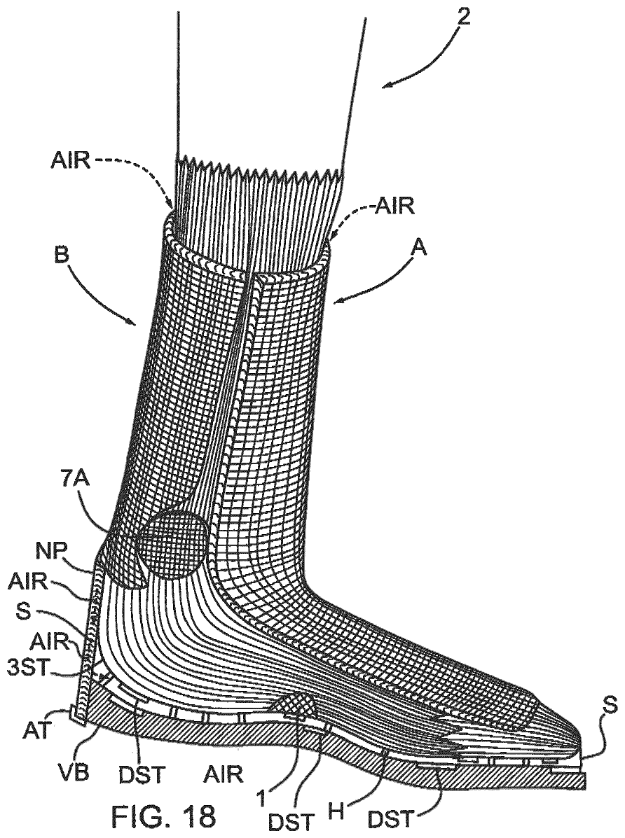

FIG. 18 shows leg 2 with a plantar ulcered wound as in as in 1 being ventilated by AIR via 30 fabric spacers as in A, B, 2A, dorsal NP ventilating 3D fabric spacer and being ventilated by holes H of sole S.

Air as in AIR penetrates the dorsal 3D fabric spacer as in B and travels downwards via venting stripe NP then it is dissipated horizontally inside the open spaces of the venting base as in VB then it goes upwards via holes H of sole S towards the plantar surface of leg 2.

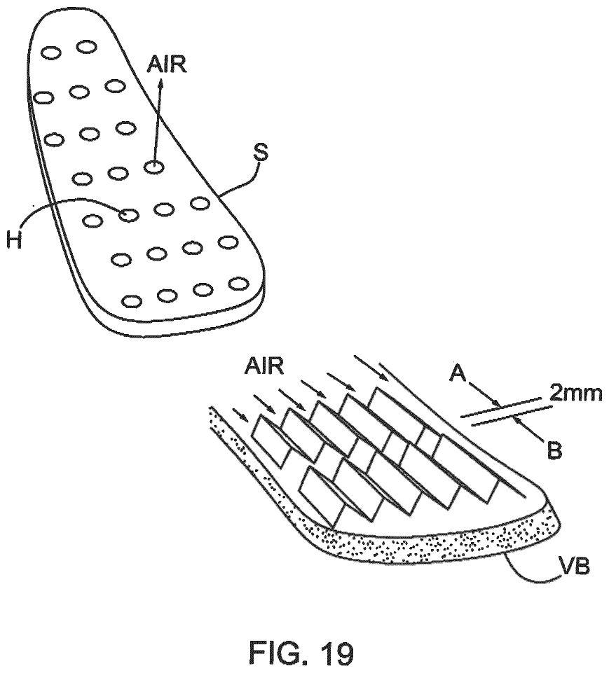

FIG. 19 shows the venting system of FIG. 18 comprising a sole as in S with vented holes with atmospheric air as in AIR.

FIG. 19 shows a venting base as in VB with sole S ready to be installed on top of it.

The VB base has pyramidal protrusions with gaps as In AB distant to each other by 2.00 mm in order to offer more flexibility during assemblies of device around and underneath the foot.

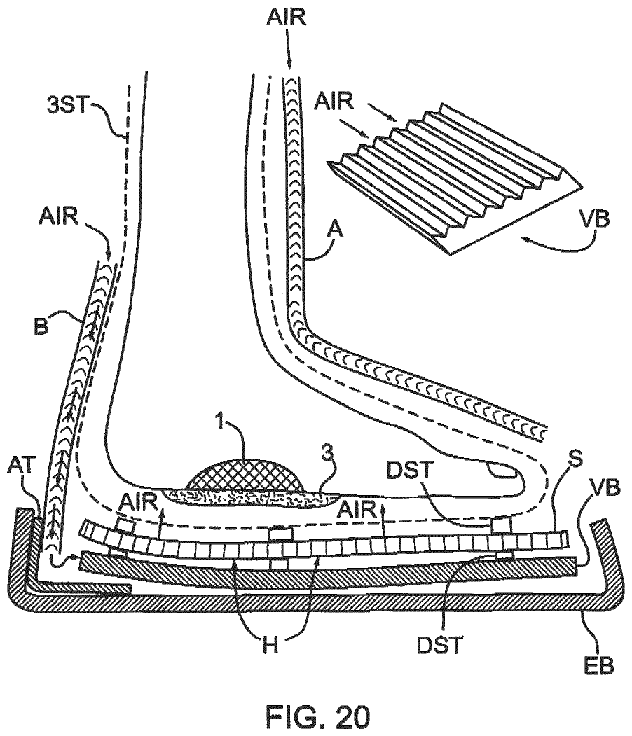

FIG. 20 depicts a wounded foot as in 1 with an additional external optional cushioning base as in EB.

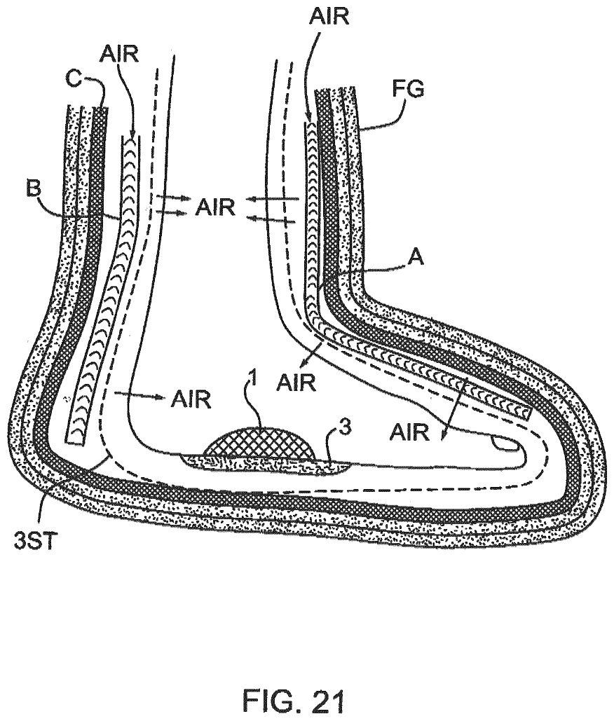

FIG. 21 depicts an aerated foot by atmospheric AIR via 3D fabric spacers as in A and B.

FIG. 21 is a total contact cast via two or three layers of fiber glass cast as in FG.

It will be appreciated that, preferably, the compressible 3D fabric spacers are not resilient meaning they remain compressed, that is in a compressed or flattened state, even after removal of the immobilizing cast.

As described hereinbefore, the present invention relates to a system or kit for a total contact cast to treat a wound 1 on a patient's leg 2, for example a diabetic foot ulcer on a sole or plantar portion of the foot. The system or kit generally comprises:
a wound dressing 3 configured for application to the wound, for example, by adhesion to skin of the patient's leg;
a fluidically permeable stockinette 3ST, in other words a sock, configured to receive the patient's leg 2 with the wound dressing 3 on the wound;
at least one ventilation member, such as A or B in FIG. 6, arranged for mounting to the stockinette 3ST; and
cast material FG for forming an outermost encapsulating layer of the total contact cast.

Each ventilation member such as A or B respectively comprises a body of three dimensional (3D) fabric spacer material forming therein a pathway for air flow from an ambient environment to the patient's leg, as denoted by AIR in FIG. 11.

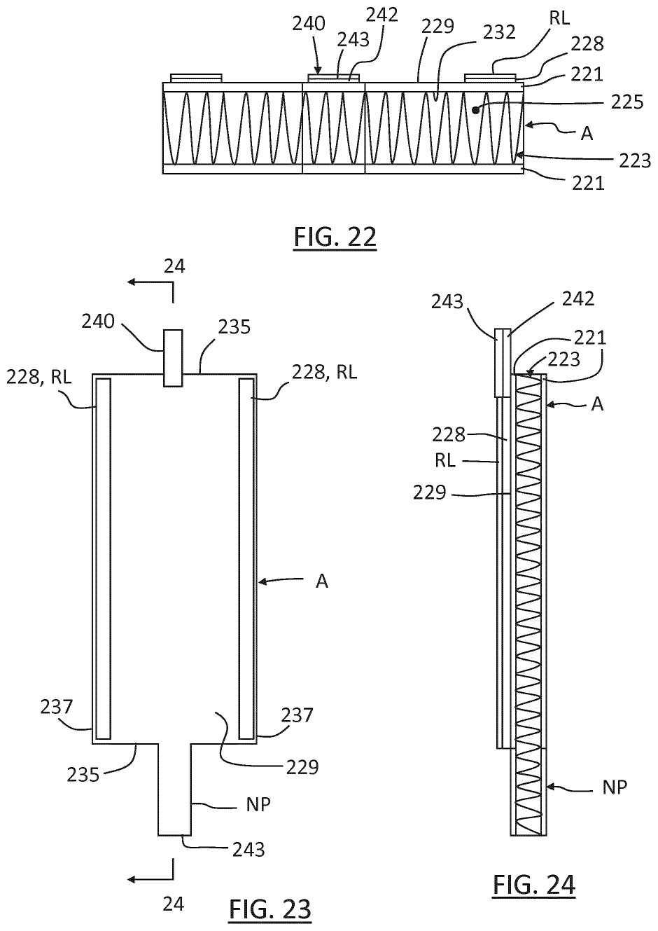

Referring to FIGS. 22-24, the body of 3D fabric spacer material of each ventilation member is of a conventional construction and comprises a pair of sheets of fabric 221 interconnected in generally parallel relation to one another by yarn or thread 223. The yarn or thread forms a web between inner faces of the generally parallel fabric sheets. Thus, in effect there is formed a sandwich-type panel of fabric, an interior 225 of which is substantially hollow besides a web of interconnecting yarn/thread 223 through which fluid, in particular air, can flow. The 3D fabric spacer material is flexible, compressible in a thickness direction between the sheets 221 and stretchable in longitudinal and transverse or lateral directions of the body, that is parallel to planes of the constituent sheets.

Each of the ventilation members A, B comprises an attachment device 228 supported on one side of the ventilation member arranged for facing the stockinette 3ST, which attachment device is configured to mount to the stockinette. More specifically, the attachment device comprises one or more adhesive areas 228 on a common side 229 of the ventilation member and removably covered, for example by removable release liners RL, for adhesively mounting to the stockinette 3ST. That is, the adhesive areas 228 comprise layers of adhesive on an outer face of the 3D fabric spacer material defining one of the sides of the ventilation member and configured for bonding to the stockinette. The 3D fabric spacer material provides a substrate for the stockinette adhesive.

Typically, an opposite side to that carrying the attaching adhesive 228 is bare, meaning it is formed by an outer face of an opposite one of the sheets of the body of 3D fabric spacer material, which is wholly exposed and uncovered.

Thus, if each of the constituent sheets 221 of a respective body of 3D fabric spacer material has an inner face 232 oriented to face an opposite one of the sheets and an outer face 229 opposite to the inner face, the adhesive 228 for bonding to the stockinette 3ST is provided on the outer face 229 of one of the fabric sheets forming a common side of the ventilation member.

The one or more adhesive areas 228 comprise a plurality of adhesive areas at spaced locations on the common side 229 of the ventilation member. When there are plural adhesive areas, a plurality thereof are located adjacent a periphery of the respective ventilation member, which is open for fluidic communication with a surrounding environment.

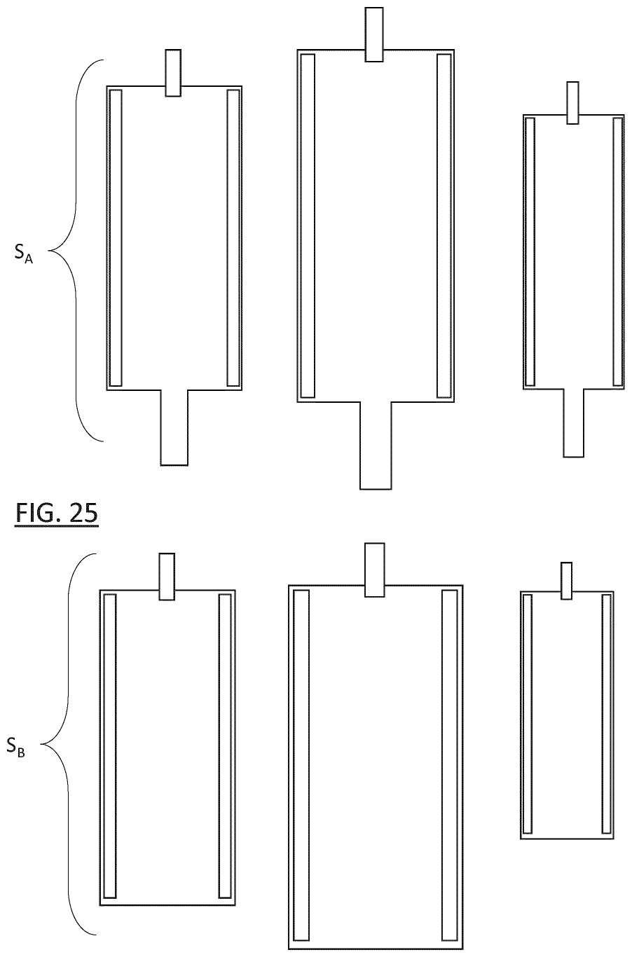

Referring to FIG. 25, when provided as a kit of parts for subsequent assembly on the patient's leg 2 to form the total contact cast, the one or more ventilation members A, B of the kit comprises at least one set of prefabricated ventilation members having a common shape and different dimensions for selective application to the patient's leg. The shape of each of the sets of the prefabricated ventilation members is based on an area of the leg to be covered thereby for ventilation or aeration. Thus, the installer is provided multiple sizes of a common type of the ventilation member associated with a prescribed area of the leg, to which the ventilation member conforms upon application, for selection of the correct size based on the patient's leg. For example, each size of the ventilation member of a common type has a prescribed combination of length and width associated with a common shape.

Typically, the ventilation members comprise plural sets having different shapes for mounting to different areas of the patient's leg. For example, a first set SA is configured, for example by being shaped, for mounting to a dorsal lower leg portion of the patient's leg and a second set SB is configured for mounting to a frontal lower leg portion, which is complementary and/or opposite to the aforementioned dorsal portion.

One of the ventilation members typically comprises a lower leg ventilation member A or B arranged to cover a lower leg portion of the patient's leg excluding a foot thereof. The lower leg ventilation member is longitudinally elongated to have longitudinally opposite ends 235 and transversely opposite sides 237 which are longer in length than the ends, so as to be arranged oriented in a lengthwise direction of the patient's leg between the foot and a hip. Since the respective ventilation member comprises 3D fabric spacer material, the ends 235 and the sides 237 defining the periphery of the respective ventilation member are open. Opposite sides 229 of the respective ventilation member are formed by the constituent sheets 221 of the material. For example, the lower leg ventilation member may be configured to cover, by conforming to, a frontal or dorsal portion of the lower leg portion of the patient's leg.

The lower leg ventilation member A or B further includes a preliminary mounting member 240 located at one of the ends 235, preferably a top one arranged to be oriented above an opposite one of the ends, and substantially transversely centrally of this end. The mounting member 240 is configured to attach to the stockinette 3ST for positioning the lower leg ventilation member in relation to the lower leg portion of the patient's leg. Preferably, the preliminary mounting member 240 is configured to attach to the stockinette by bonding, and thus comprises removably covered adhesive, including an adhesive base layer 242 (with adhesive on one side or face thereof) and a removable cover or liner 243, similarly to the ventilation member. For example, the preliminary mounting member 240 is in the form of a tab projecting from the end 235 of the ventilation member at which it is located, and it is attached to the ventilation member on a common side 229 as the adhesive areas 228 where the member bondingly and securely mounts to the stockinette.

The adhesive 228 for securing to the stockinette is larger in area than the preliminary mounting member and covers a larger area of the common side 229 of the ventilation member in comparison thereto. Furthermore, the stockinette adhesive is provided at both ends and at both sides of the ventilation member, which in the illustrated arrangement is achieved by a pair of longitudinally extending, elongated strips of adhesive located adjacent the sides 237. In the illustrated arrangement, there is no adhesive intermediate the opposite strips except for that of the preliminary mounting member which does not overlap the strips in the longitudinal direction of the ventilation member.

In the case of the lower leg ventilation member, this may be arranged to cover a dorsal portion of the lower leg portion, meaning a back of the leg, between a backside of the knee and the foot, as indicated at A. In this case, the ventilation member includes an integral tailpiece NP at a bottom one of the ends 235 arranged to be in proximal relation to the foot. The tailpiece NP is elongated relative to a longitudinal direction of the lower leg dorsal ventilation member from the bottom end 235 to a distal end 243 of the tailpiece, and the tailpiece is narrower in width relative to a transverse direction than the body of 3D fabric spacer material forming the lower leg dorsal ventilation member. The tailpiece NP is integrally formed with the ventilation member so as to be formed from a common body of 3D spacer fabric material. The tailpiece thus serves as an extension of the air pathway formed by the hollow interior of the ventilation member, but is narrower so as not to obstruct other components of the total contact cast that may be located around an ankle or malleoli of the foot while still delivering ambient air to the foot, preferably the sole thereof. Thus, in an installed position of the lower leg dorsal ventilation member, the tailpiece registers with a back of the ankle.

There is also disclosed herein a method for forming a total contact cast to treat a wound 1 on a patient's leg 2 which generally comprises the steps of:
applying a wound dressing 3 to the wound 1;
after applying the wound dressing 3, covering the patient's leg with a stockinette 3ST;
after covering the patient's leg with a stockinette, mounting one or more ventilation members such as A, B respectively comprising three dimensional (3D) fabric spacer material to the stockinette 3ST; and
after mounting one or more ventilation members, encapsulating in cast material such as FG.

Preferably, mounting one or more ventilation members to the stockinette comprises bonding each of the ventilation members to the stockinette.

Preferably, before or as part of the step of mounting the one or more ventilation members to the stockinette, there is provided a step of selecting, for each area of the patient's leg to be aerated, one of a plurality of ventilation members of a prefabricated set with a common shape arranged to conform to a prescribed portion of the patient's leg and different dimensions, such as from set SA.

Optionally, mounting one or more (selected) ventilation members to the stockinette comprises mounting at least one of ventilation member in a stretched condition over protruding portions of the patient's leg.

Preferably, for ease of application, mounting one or more (selected) ventilation members to the stockinette comprises respectively aligning the ventilation members relative to the patient's leg using a first adhesive portion 240 at one end 235 of the respective ventilation members and, after aligning, respectively securing the ventilation members using a second adhesive portion 228 covering a larger surface area of the respective ventilation member than the first adhesive portion.

This provides an arrangement of total contact cast which can be more quickly and simply formed or assembled.

This arrangement provides a quicker and simpler manner of forming a total contact cast by bonding using existing adhesive applied to the ventilation member, that is a premanufactured component, instead of using fastening mechanisms or devices distinct from the ventilation member to secure the same to the patient's leg.

This arrangement provides a quicker and simpler manner of forming a total contact cast by provision of prefabricated components from which suitable components may be selected. The scope of the claims should not be limited by the preferred embodiments set forth in the examples but should be given the broadest interpretation consistent with the specification as a whole.

1. A system for a total contact cast to treat a wound on a patient's leg comprising: a wound dressing configured for application to the wound;
a fluidically permeable stockinette configured to receive the patient's leg with the wound dressing on the wound;
at least one ventilation member arranged for mounting to the stockinette, wherein said at least one ventilation member respectively comprises a body of three dimensional (3D) fabric spacer material forming therein a pathway for air flow from an ambient environment to the patient's leg; and
cast material for forming an outermost encapsulating layer of the total contact cast; wherein the at least one ventilation member respectively comprises one or more adhesive areas on a common side of the ventilation member and removably covered for adhesively mounting to the stockinette.

2. The system of claim 1 wherein, when the at least one ventilation member respectively comprises a lower leg ventilation member arranged to cover a lower leg portion of the patient's leg excluding a foot thereof, and when the lower leg ventilation member is longitudinally elongated to have longitudinally opposite ends and transversely opposite sides which are longer in length than the ends, so as to be arranged oriented in a lengthwise direction of the patient's leg between the foot and a hip, the lower leg ventilation member further includes a preliminary mounting member located at one of the ends and substantially transversely centrally thereof, wherein the preliminary mounting member is configured to attach to the stockinette for positioning the lower leg ventilation member in relation to the lower leg portion of the patient's leg.

3. The system of claim 2 wherein the preliminary mounting member comprises removably covered adhesive so as to be configured to attach to the stockinette by bonding.

4. The system of claim 1 to 3 wherein the at least one ventilation member comprises a selected one of a plurality of prefabricated ventilation members having a common shape and different dimensions.

5. The system of claim 1 to 4 wherein, when the at least one ventilation member respectively comprises a lower leg dorsal ventilation member arranged to cover a dorsal lower leg portion of the patient's leg between the knee and the foot of the patient's leg, and when the lower leg dorsal ventilation member is longitudinally elongated to have longitudinally opposite ends and transversely opposite sides which are longer in length than the ends, the lower leg dorsal ventilation member further includes an integral tailpiece at a bottom one of the ends arranged to be in proximal relation to the foot, wherein the tailpiece is elongated relative to a longitudinal direction of the lower leg dorsal ventilation member from the bottom end to a distal end of the tailpiece, and wherein the tailpiece is narrower in width relative to a transverse direction than the body of 3D fabric spacer material forming the lower leg dorsal ventilation member.

6. A kit for forming a total contact cast to treat a wound on a patient's comprising: a wound dressing configured for application to the wound;
a fluidically permeable stockinette configured to receive the patient's leg with the wound dressing on the wound;
at least one aeration arranged for mounting to the stockinette, wherein said at least one ventilation member respectively comprises a body of three dimensional (3D) fabric spacer material forming therein a pathway for air flow from an ambient environment to the patient's leg; and cast material for forming an outermost encapsulating layer of the total contact cast; wherein the at least one ventilation member respectively comprises an attachment device supported on one side of the ventilation member and configured to mount to the stockinette;
wherein the at least one ventilation member comprises at least one set of prefabricated ventilation members having a common shape and different dimensions for selective application to the patient's leg.

7. The kit of claim 6 wherein the at least one set of prefabricated ventilation members comprises a plurality of the sets having different shapes for mounting to different areas of the patient's leg.

8. The kit of claim 6 or 7 wherein the at least one ventilation member respectively includes one or more adhesive areas on a common side of the ventilation member and removably covered for adhesively mounting to the stockinette.

9. The kit of claim 6 wherein, when at least one of the at least one set of prefabricated ventilation members is arranged to cover a lower leg portion of the patient's leg excluding a foot thereof, and when said at least one of the at least one set of prefabricated ventilation members respectively are longitudinally elongated to have longitudinally opposite ends and transversely opposite sides which are longer in length than the ends, so as to be arranged to be oriented in a lengthwise direction of the patient's leg between the foot and a hip, each of the ventilation member further includes a preliminary mounting member located at one of the ends and substantially transversely centrally thereof, wherein the preliminary mounting member is configured to attach to the stockinette for positioning said ventilation member in relation to the lower leg portion of the patient's leg.

10. The kit of claim 9 wherein the preliminary mounting member comprises removably covered adhesive so as to be configured to attach to the stockinette by bonding.

11. The kit of claim 6 wherein, when at least one of the at least one set of prefabricated ventilation members is arranged to cover a dorsal lower leg portion of the patient's leg between the knee and the foot of the patient's leg, and when said at least one of the at least one set of prefabricated ventilation members respectively are longitudinally elongated to have longitudinally opposite ends and transversely opposite sides which are longer in length than the ends, each of said at least one of the at least one set of prefabricated ventilation members further includes an integral tailpiece at a bottom one of the ends arranged to be in proximal relation to the foot, wherein the tailpiece is elongated relative to a longitudinal direction of the lower leg dorsal ventilation member from the bottom end to a distal end of the tailpiece, and wherein the tailpiece is narrower in width relative to a transverse direction than the body of 3D fabric spacer material forming the lower leg dorsal ventilation member.

12. A method for forming a total contact cast to treat a wound on a patient's leg comprising:
applying a wound dressing to the wound;
after applying the wound dressing, covering the patient's leg with a stockinette; after covering the patient's leg with a stockinette, mounting one or more ventilation members respectively comprising three dimensional fabric spacer material to the stockinette; and after mounting one or more ventilation members, encapsulating in cast material; wherein mounting one or more ventilation members to the stockinette comprises bonding each of the ventilation members to the stockinette.

13. The method of claim 12 wherein mounting one or more ventilation members to the stockinette comprises selecting each of the ventilation members from a prefabricated set having a common shape arranged to conform to a prescribed portion of the patient's leg and different dimensions.

14. The method of claim 12 wherein mounting one or more ventilation members to the stockinette comprises mounting at least one of said one or more ventilation members in a stretched condition over protruding portions of the patient's leg.

15. The method of claim 12 wherein mounting one or more ventilation members to the stockinette comprises respectively aligning said one or more ventilation members relative to the patient's leg using a first adhesive portion at one end of a respective one of the ventilation members and, after aligning, respectively securing said one or more ventilation members using a second adhesive portion covering a larger surface area of the respective ventilation member than the first adhesive portion.

16. A method for forming a total contact cast to treat a wound on a patient's leg comprising:
applying a wound dressing to the wound;
after applying the wound dressing, covering the patient's leg with a stockinette; selecting, for each area of the patient's leg to be aerated, one of a plurality of ventilation members of a prefabricated set with a common shape arranged to conform to a prescribed portion of the patient's leg and different dimensions;
after covering the patient's leg with a stockinette, mounting the selected ventilation member to the stockinette; and
after mounting the selected ventilation member, encapsulating in cast material.

17. The method of claim 16 wherein mounting one or more ventilation members to the stockinette comprises bonding each of the ventilation members to the stockinette.

18. The method of claim 16 wherein mounting one or more ventilation members to the stockinette comprises mounting at least one of said one or more ventilation members in a stretched condition over protruding portions of the patient's leg.

19. The method of claim 16 wherein mounting the selected ventilation member to the stockinette comprises aligning the selected ventilation member relative to the patient's leg using a first adhesive portion at one end of the selected ventilation members and, after aligning using the first adhesive portion, securing the selected ventilation member using a second adhesive portion covering a larger surface area of the selected ventilation member than the first adhesive portion.
